(19) United States
(12) Patent Application Publication
(10) Pub. No.: US 2011/0301236 A1
(43) Pub. Date: Dec. 8, 2011

(54) HeLa-C3 Apoptosis Inducer from Garcinia paucinervis

(76) Inventors: XUE-MEI GAO, Hong Kong (CN); Ting Yu, Singapore (SG); Kathy Qian Luo, Singapore (SG); Hong Xi Xu, Hong Kong (CN)

(21) Appl. No.: 12/793,690

(22) Filed: Jun. 4, 2010

(51) Int. Cl. A61K 31/235; A61P 35/00; C07C 69/94

(52) U.S. Cl. 514/544; 560/70

(57) ABSTRACT

A novel compound isolated from the plant Garcinia paucinervis. The compound's structure is as follows:

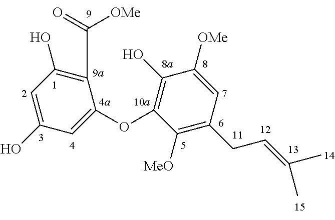

The compound possesses a potent ability to activate caspase-3 in HeLa-C3 cells within 72 h at a low concentration of 25 μM. The IC₅₀ on HeLa cells for this compound was measured to be below 10 μM, indicating a strong HeLa cell growth inhibitory effect.

FIELD OF THE INVENTION

This invention relates to new chemical entity isolated from natural sources for their therapeutic uses. More particularly, it relates to a compound that is naturally occurring in the plant of Garcinia paucinervis and its biological activity of inducing apoptosis of tumor cells and inhibitory effect on tumor cell growth.

### BACKGROUND OF THE INVENTION

Apoptosis is a genetically programmed and physiological mode of cell death that leads to the removal of unwanted or abnormal cells. Effective cancer therapeutic strategies often rely on preferential and efficient induction of apoptosis in tumor cells. Caspase-3 protease plays important roles in the signaling pathway controlling mammalian apoptosis. Natural products which can activate caspase-3 are functionally important in the induction of apoptosis and represent a type of bioactive natural products.

While with today's high throughput chemical synthetic technologies and high efficiency screening methodologies it may be easy to find a large number of chemical compounds that shows promising biological effect at cellular levels in the laboratory, it appears that the rate of these compounds becoming clinically useful is very low due to a number of factors. One of the factors is their toxicity, which are often found to be to too severe to be tolerable by human body at a later stage of the new drug development. In this respect, new compounds discovered from natural sources which have been used as medicines for thousands of years are believed to hold advantages because they have been consumed by human for a long time and their toxicity therefore are more likely to be tolerable than purely synthetic compounds.

### SUMMARY OF THE INVENTION

The tropical genus Garcinia is well known to be a rich source of bioactive isoprenylated xanthones and benzophenones. As part of phytochemical and pharmacological investigations of Garcinia plants in China, a bio-guided isolation of the G. paucinervis leaves was conducted in the present invention, which has led to the isolation of 19 compounds, four of which were previously unknown.

One object of the present invention is to provide a new natural occurring compound having the effect of inducing apoptosis and are useful for developing into anti-cancer drugs. This objective was achieved by isolating a new chemical compound from Garcinia plants which has such effect on apoptosis. The novel compound, referred to as Compound 2 in this application, has a structural below:

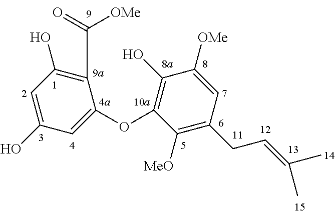

Another object of the present invention is to provide a method of inducing apoptosis in tumor cells. This object is achieved by administering to a mammalian subject a therapeutically effective amount of the compound defined above.

Still another object of the present invention is to provide a pharmaceutical composition for treating cancers. This object is achieved by formulating a pharmaceutical composition with a compound as defined above and a pharmaceutically acceptable carrier and other suitable additives by a conventional method known in the pharmaceutical industry. The pharmaceutical composition is preferably formulated in a dosage form selected from the group consisting of tablet, capsule, and injection.

The various features of novelty which characterize the invention are pointed out with particularity in the claims annexed to and forming a part of this disclosure. For a better understanding of the invention, its operating advantages, and specific objects attained by its use, reference should be made to the drawings and the following description in which there are illustrated and described preferred embodiments of the invention.

### BRIEF DESCRIPTION OF THE DRAWINGS

FIG. 1 shows the selected HMBC (→) and ¹H-¹H COSY (—) correlations of compounds 1 and 2;

FIG. 2 depicts cell morphology changes during the course of compound treatment with the compound of the present invention (i.e., compound 2).

FIG. 3 shows the viability of the cells 72 hours following treatment with compounds 2 at 25 μM in comparison with paclitaxol.

### DETAILED DESCRIPTION OF PARTICULAR EMBODIMENTS OF THE INVENTION

### Experimental Procedures

Optical rotations were measured with a JASCO DIP-1000 polarimeter. Ultraviolet absorption spectra were recorded using a Perkin-Elmer Lambda L14 spectrometer. A Perkin Elmer spectrum 100 FT-IR spectrometer was used for scanning IR spectroscopy with KBr pellets. 1D and 2D NMR spectra were recorded on a Bruker AV-400 spectrometer with TMS as internal standard. Chemical shifts (d) are expressed in ppm with reference to the solvent signals. HRMS were obtained using a nanoLC-MS/MS system, with a nanoAcquity HPLC module and a Q-TOF spectrometer equipped with a nanoelectrospray ion source (Waters, Milford, Mass.) and provided by a lock-mass apparatus to perform a real-time calibration correction. Column chromatography was performed with silica gel (200-300 mesh, Qingdao Marine Chemical, Inc., Qingdao, People's Republic of China), Sephadex LH-20 (Pharmacia), and reversed-phase C18 silica gel (250 mesh, Merck). Precoated TLC sheets of silica gel 60 GF₂₅₄ were used. An Agilent 1100 series equipped with an Alltima C18 column (4.6×250 mm) was used for HPLC analysis, and semipreparative and preparative Alltima C18 columns or Zorbax SB-C18 columns (9.4×250 mm and 22×250 mm) were used in sample preparation. Spots were visualized by heating silica gel plates sprayed with 10% H₂SO₄ in EtOH.

### Plant Material

The stems of G. paucinervis were collected in October 2008 from Xishuangbanna Prefecture of Yunnan Province, China. The plant was identified by Pan-Yu Ren. A voucher specimen (CMED-047404) has been deposited at Hong Kong Jockey Club Institute of Chinese Medicine.

### Extraction and Isolation

Air-dried and powdered leaves (2.8 kg) were extracted with Me₂CO (3×15 L) at room temperature and concentrated in vacuo to give a crude extract, which was partitioned between H₂O and CH₂Cl₂. The CH₂Cl₂-soluble portion (182 g) was decolourized by MCI. The 90% methanol part (57 g) was chromatographed on a silica gel column eluting with hexane-acetone (1:0, 40:1, 9:1, 8:2, 7:3, 1:1 and 0:1) to afford five fractions, I-V.

Fraction I (6 g) was performed on reversed-phase column (RP-18) eluting with MeOH/H₂O (30%-90%) to give 23 fractions. Fraction I-11 (587 mg), 1-14 (234 mg), I-15 (85 mg), 1-18 (43 mg) were separately subjected to semi-preparative HPLC (MeOH—H₂O, 60:40) to yield compound 17 (75 mg), 18 (8 mg), 4 (62 mg), 16 (9 mg), 19 (7 mg), (7 mg), 6 (6 mg). Fraction II-1 (7 g) (the first part of fraction II) was then performed on reversed-phase column (RP-18) eluting with MeOH/H₂O (80%-100%) to give 17 fractions. Fraction II-1-7, II-1-9, II-1-10 were separated over Sephadex LH-20 eluting with MeOH and then subjected to semi-preparative HPLC (MeOH—H₂O, 80:20) to yield compounds 3 (10 mg), 11 (3 mg), 2 (4 mg), 12 (5 mg), 1 (3 mg), 15 (4 mg). Fraction II-2 (4 mg) was separated over Sephadex LH-20 eluting with MeOH and then subjected to semi-preparative HPLC (MeOH—H₂O, 60:40) to yield compounds 7 (2 mg), 13 (3 mg), 14 (2 mg), 8 (6 mg) and 9 (4 mg).

Paucinervin A (which is compound 1): yellow gum; [α]²⁴-3.7 (c 0.61, MeOH); UV (MeOH) λ max (log ε) 278 (2.25), 250 (1.98), 220 (2.55) nm; IR (KBr) ν max 3411, 2924, 1624, 1493, 1465, 1424, 1384, 1283, 1201, 1146, 1069, 982 cm⁻¹; ¹H and ¹³C NMR data, Table 1; positive HRESIMS m/z 427.1694 [M+H]⁺ (calcd 427.1757 for C₂₄H₂₇O₇).

Paucinervin B (which is compound 2): brown gum; [a]²⁴-3.8 (c 0.26, MeOH); UV (MeOH) λ max (log ε) 267 (2.36), 245 (2.04), 225 (2.71) nm; IR (KBr) ν max 3411, 2928, 1621, 1438, 1370, 1320, 1265, 1193, 1114, 1069 cm⁻¹; ¹H and ¹³C NMR data, Table 1; positive HRESIMS m/z 405.1540 [M+]⁺ (calcd 405.1549 for C₂₁H₂₅O₈).

### Bioassay

The bioassay method was described in our previous paper with some modifications. All the testing samples were dissolved in DMSO to make stock solutions. The concentration of each stock was at least 1000 times higher than the working concentration. HeLa-C3 cells, which allows detection of apoptotic cell death involving caspase activation, were cultured in minimum essential medium (MEM) containing 10% fetal bovine serum, 100 U/mL penicillin, 100 mg/mL streptomycin, in a 5% CO2 humidity incubator at 37° C. The sample well for apoptotic activity testing was prepared by seeding a well on a 96-well plate with 7500 HeLa-C3 cells suspended in 100 μL culture medium. After 12-16 h incubation, the old medium was removed and 100 μL freshly prepared culture medium containing the testing sample at a certain working concentration was added to both the sample well and the corresponding background well. Culture medium containing 0.1% DMSO was the negative control while 500 nM paclitaxel was the positive control. After that, the plate was read repeatedly by a Perkin-Elmer Victor reader with excitation wavelength at 440±10 nm and emission wavelength at 486±8 nm for CFP (cyan fluorescent protein) and 535±8 nm for YFP (yellow fluorescent protein) at indicated time points. The data acquisition duration was up to 72 h. The YFP/CFP emission ratio was then calculated. The background fluorescence was measured from the wells containing only medium. After subtracting the background fluorescence from the recorded signal, net YFP and CFP readings were obtained. In this paper, Y/C emission ratio is used to represent the effect of FRET, which is equal to the net YFP reading divided by the net CFP reading from the same well. If YFP/CFP emission ratio was reduced below 3, the testing sample was considered as a good apoptotic inducer at that concentration. All samples were tested in triplicate. The whole experiment was repeated for three times.

The measurement of the IC₅₀ was conducted on the isolated compounds using MTT assay. MIT powder was dissolved in PBS at a concentration of 5 mg/mL. For MTT assay, 10 μL of MTT solution was added into each well of a 96-well plate. After 2 h incubation at 37° C., 100 μL 10% SDS solution with 0.01 M HCl was added to dissolve the purple crystals. After 24 h incubation, the optical density (OD) readings at 595 nm were measured using a plate reader. Firstly, 2,500 HeLa cells suspended in 100 μL MEM medium were seeded respectively in a 96-well plate. After 24 h incubation, fresh medium contains various concentrations of each compound were added into the 96-well plate and changed the old medium. The concentrations applied except compound 1 which was from 200 μM to 3.125 μM were ranged from 100 μM to 1.5625 μM, which was achieved by doing two-fold dilutions for 6 times. The OD values of the control group at 0 h and 72 h together with the compound treated groups at 72 h from the MTT assay were measured using a plate reader. IC50 is the concentration of a compound inhibiting 50% of the cell growth.

### The New Compounds

Paucinervin A (Compound 1) was obtained as a yellow gum. Its molecular formula was established as C₂₄H₂₆O₇ by HRESIMS at m/z 427.1694 [M+H]⁺, suggesting twelve degrees of unsaturation. The ¹H and ¹³C NMR data of 1 (Table 1) showed the presence of five methyls, two methylenes, four olefinic methines, one

TABLE 1
¹H and ¹³C NMR Data for Paucinervins A and B (i.e., Compound 1 and 2)

| | 1^{b} | | | | 2^{c} | | | |
|---|---|---|---|---|---|---|---|---|
| No. | δ_{C} | | δ_{H} | | δ_{C} | | δ_{H} | |
| 1 | 162.8 | s | | | 164.7 | s | | |
| 2 | 111.3 | s | | | 95.0 | d | 5.56 | (d, 2.3) |
| 3 | 162.0 | s | | | 162.6 | s | | |
| 4 | 100.5 | d | 6.35 | (s) | 97.6 | d | 5.96 | (d, 2.3) |
| 4^{a} | 159.4 | s | | | 165.7 | s | | |
| 5 | 137.8 | s | | | 145.6 | s | | |
| 10^{a} | 140.6 | s | | | 133.7 | s | | |
| 6 | 144.8 | s | | | 124.0 | s | | |
| 7 | 124.8 | s | | | 112.4 | d | 6.50 | (s) |
| 8 | 115.6 | d | 6.79 | (s) | 147.6 | s | | |
| 8^{a} | 137.6 | s | | | 134.8 | s | | |
| 9 | 168.5 | s | | | 172.6 | s | | |
| 9^{a} | 98.7 | s | | | 97.8 | s | | |
| 11 | 22.0 | t | 3.38 | (d, 7.2) | 29.2 | t | 3.26-3.29 | (m) |
| 12 | 120.9 | d | 5.19-5.26 | (m) | 124.0 | d | 5.22-5.27 | (m) |
| 13 | 136.0 | s | | | 133.4 | s | | |
| 14 | 17.9 | q | 1.80 | (s) | 25.9 | q | 1.73 | (s) |
| 15 | 25.7 | q | 1.74 | (s) | 17.8 | q | 1.74 | (s) |
| 16 | 27.6 | t | 3.27 | (d, 7.4) | | | | |
| 17 | 120.8 | d | 5.19-5.26 | (m) | | | | |
| 18 | 134.1 | s | | | | | | |
| 19 | 17.7 | q | 1.68 | (s) | | | | |
| 20 | 25.7 | q | 1.74 | (s) | | | | |
| OMe | 62.6 | q | 4.08 | (s) | 61.5 | q | 3.77 | (s) |
| OMe | | | | | 61.4 | q | 3.74 | (s) |
| COOMe | | | | | 52.4 | q | 3.89 | (s) |
| OH-1 | | | 11.32 | (s) | | | | |

^{a}Data were recorded with a Broker DRX-400 MHz spectrometer, chemical shifts (δ) are in ppm, J in Hz; assignments were confirmed by ¹H-¹H COSY, HMQC and HMBC.
^{b}Data were recorded in CHCl_{3,}
^{c}Data were recorded in CD₃OD.

carbonyl carbon and twelve olefinic quaternary carbons. The IR spectrum showed absorption bands for hydroxyl groups (3411 cm⁻¹) and a lactone carbonyl group chelated to an ortho-hydroxyl group (1624 cm⁻¹). The presence of the latter functionality was confirmed by resonances at δ_{C} 168.5 (C-9) and δ_{H} 11.32 (OH-1). The NMR spectra showed two sets of signals corresponding to prenyl groups. The foregoing data indicated that 1 was a depsidone derivative that contained two isoprene units.

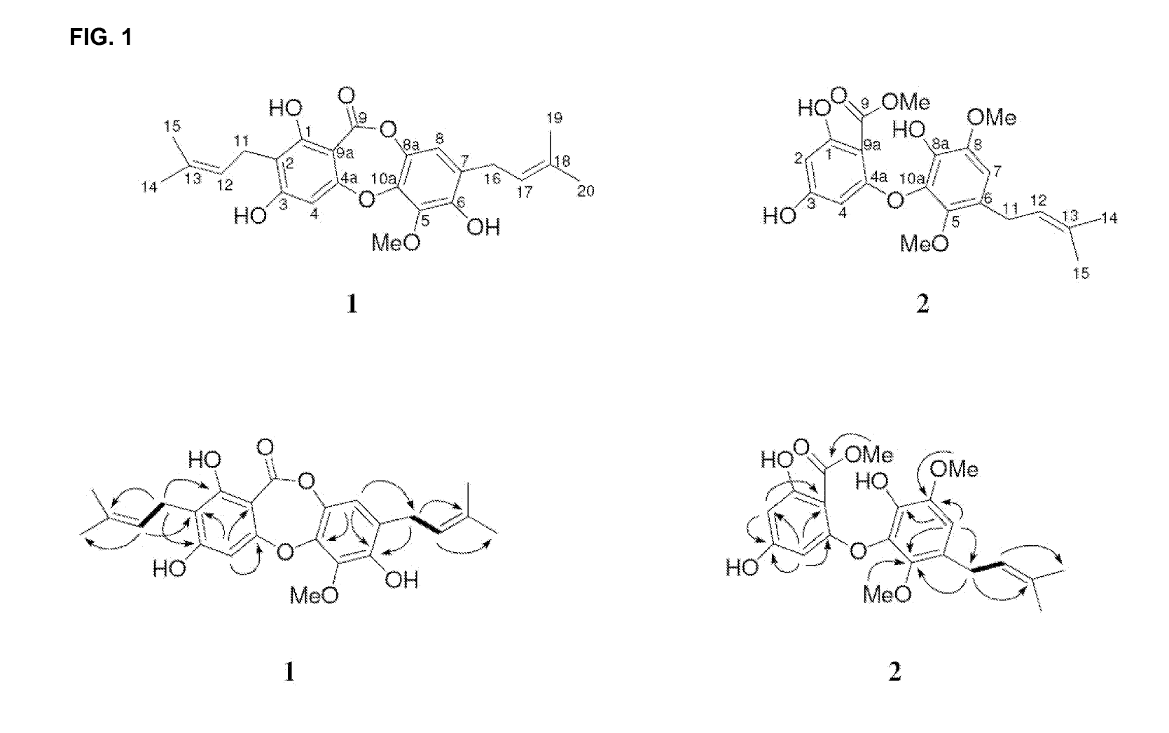

The analysis of 2D NMR spectra using HMQC and HMBC techniques enabled the assignment of ¹H and ¹³C NMR signals (see FIG. 1). Since the NMR data of 1 were similar to those of the known parvifolidone B¹⁸, the possible structure was established by a detailed comparison of its NMR data with those of this known compound, and suggested the same core structure of both compounds. In the HMBC spectrum, the chelated proton signal at δ_{H} 11.32 corresponding to OH-1 showed a strong cross-peak with the carbon at δ_{C} 98.7 (C-9a), which should be the aromatic carbon linked to the carbonyl group. The OH-1 proton also exhibited connectivity to δ_{C} 162.8 (oxygenated aromatic carbon C-1) and δ_{C} 111.3 (substituted aromatic carbon C-2). The cross-peaks of C-2/(H-11, -12) confirmed that C-2 was substituted with a prenyl group. The aromatic proton at δ_{H} 6.35 (C-4) showed connectivity to four aromatic carbons at δ_{C} 98.7 (C-9a), 159.4 (C-4-a), 162.0 (C-3), and 111.3 (C-2). The second prenyl group was determined to be at C-7 based on the connectivity of the carbon at δ_{C} 124.8 (C-7) to H-16 and H-17. A methoxy group resonating at δ_{H} 4.08 in the ¹H NMR spectrum of 1 was located at C-5 according to its HMBC correlation with C-5. The quaternary carbon signal of δ_{C} 144.8 (C-6), 162.0 (C-3) and C-6 and molecular formula C₂₄H₂₆O₇ indicated the presence of two hydroxyl groups at C-3 and C-6, respectively. Thus, compound 1, identified as a new compound, was named paucinervin A.

Paucinervin B (compound 2) was obtained as a brown gum. The molecular formula of the compound was determined to be C₂₁H₂₄O₈ by HRESIMS at m/z 405.1540 [M+H]⁺. It exhibited UV and IR absorption bands similar to those of 1. Comparison of the NMR data between 2 and 1 indicated that they are different in the substitutes on the aromatic rings. The presence of one prenyl group was deducted from the NMR spectra. The prenyl group was located at C-6 of the aromatic ring in 2 based on correlations of H₂-11 to C-5, C-6 and C-7. Two methoxy groups were located at C-5 (δ_{C} 145.6) and C-8 (δ_{C} 147.6) on the basis of their correlations with these two carbon signals, respectively. Resonances for the singlet aromatic proton and one of the prenyl groups in 1 were replaced by signals of two metaaromatic protons [δ_{H} 5.56 (d, J=2.3 Hz) and 5.96 (d, J=2.3 Hz)] in 2. The higher field aromatic proton was attributed to H-2 according to its HMBC correlations with C-1 (δ_{C} 164.7), C-4 (δ_{C} 97.6), and C-9a (δ_{C} 97.8). The other meta-aromatic proton was then located at C-4. The third methoxy group (δ_{H} 3.89, δ_{C} 52.4) was located at C-9 according to HMBC correlation. The C¹³ NMR signal of C-9 (δ_{C} 172.6), appearing in lower field in ¹³C NMR spectrum than that of C-9 in 1, confirmed that the ester group between C-8a and C-9a was broken in 2. The quaternary carbon signal of δ_{C} 164.7 (C-1), 162.6 (C-3), 134.8 (C-8a) and molecular formula C₂₁H₂₄O₈ indicated the presence of three hydroxyl groups at C-1, C-3 and C-8a, respectively. Therefore, the structure of 2 wa established as shown.

### Biological Activity

The biological activity of these new compounds was evaluated for apoptosis inducing effects using genetically engineered HeLa-C3 cells that can produce a fluorescent biosensor capable of detecting caspase-3 activation. These cells emit a green light under normal growth conditions and change to a blue light when caspase-3 is activated during apoptosis to cleave the sensor protein inside the cells. This color change allows one to use a fluorescent plate reader to directly detect the activation level of caspase-3 in HeLa-C3 cells during the course of the compound treatment in a noninvasive way. Based on our previous test results, the emission ratio of YFP (yellow fluorescent protein)/CFP (cyanfluorescent proteins) is usually between 6 and 8 in normal cells, and this ratio will decrease to a value below 3 if a compound can activate caspase-3 and kill cancer cells. Therefore, any compound that can reduce the YFP/CFP emission ratio to a value below 3 is considered positive in activating apoptosis.

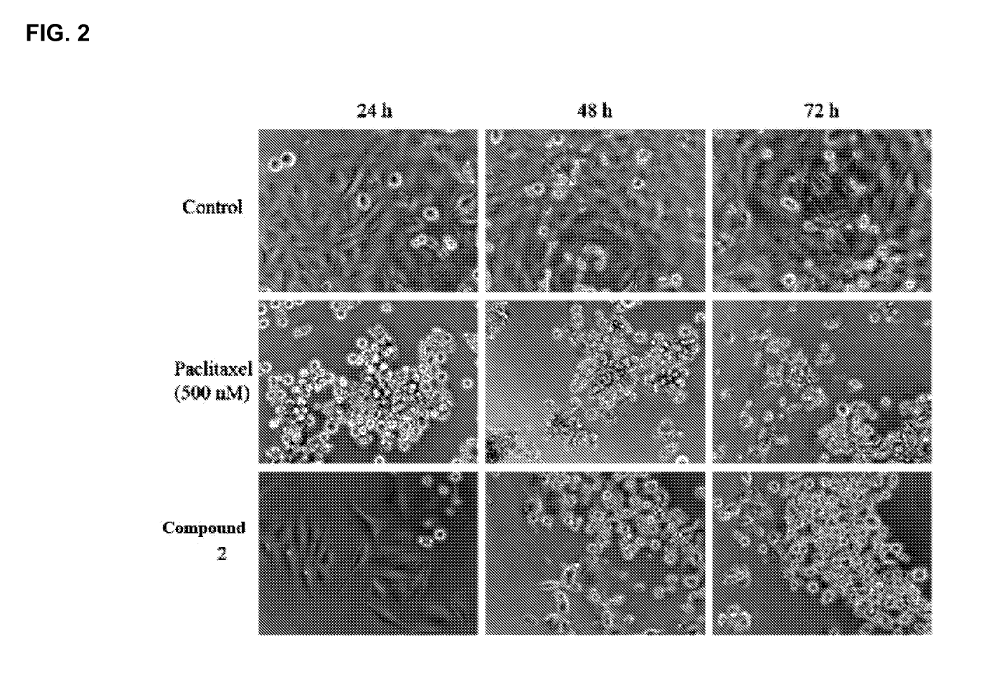

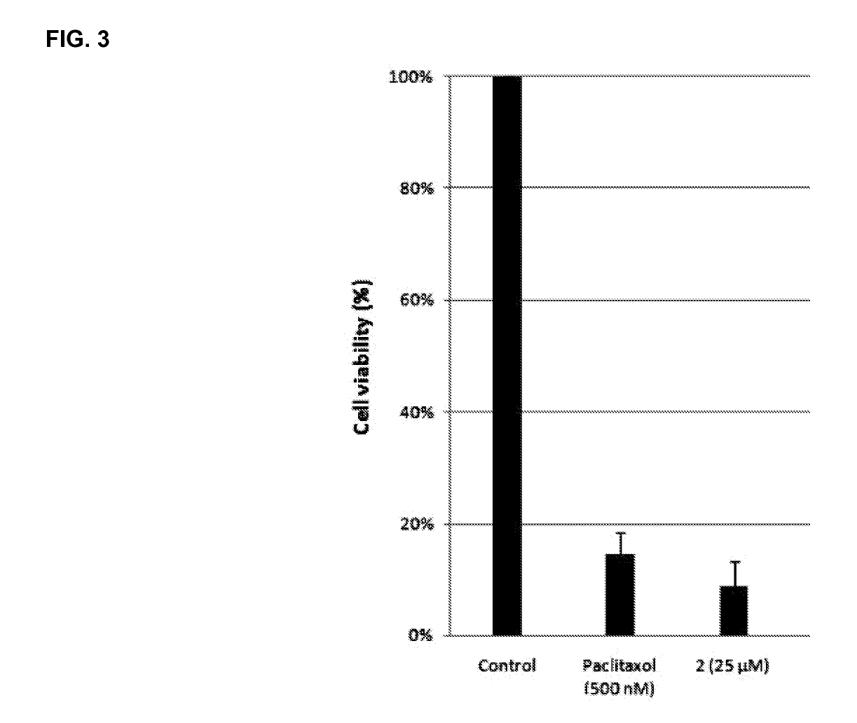

Refereeing to FIG. 2, Cell morphology changes during the course of compound treatment. HeLa-C3 cells were treated with compounds of 2 at 25 μM, an anticancer drug, pacilitaxel at 500 nM (serving as a positive control), and a control without any drug for 24, 48, and 72 h. Various cell morphologies were recorded at the indicated time points. FIG. 2 shows that the control cells have normal attached cell morphology through the course of the experiment. It further shows that cells have shrinkage and detached morphology when they were treated with either the anticancer drug paclitaxel or compound 2, confirming the compound's ability to kill cancer cells. FIG. 3 furthers shows cell viability after drug treatment 72 h.

As shown in Table 3, compounds were tested at a concentration of 100 μM, 50 μM, 25 μM and 10 μM. Among these compounds, Compound 2 (also referred to as gpl-26) could activate caspase-3 in HeLa-C3 cells within 72 h at a low concentration of 25 μM, significantly more potent than Compound 1 (also referred to as gpl-15).

TABLE 3
Apoptosis-Inducing Effects at 72 h

Apoptotic effect at

| compound | 100 μM | 50 μM | 25 μM | 10 μM |
|---|---|---|---|---|
| 1 gpl-15 | + | − | − | − |
| 2 gpl-26 | + | + | + | − |

“+” means the YFP/CFP emission ratio of compound treated HeLa-C3 cells was below 3 at 72 h. “−” means the YFP/CFP emission ratio of compound treated HeLa-C3 cells was above 3 at 72 h.

To determine the cytotoxicity of the new compounds, their IC₅₀ on HeLa cells was measured. As the results shown in the Table 4, the IC₅₀ of Compound 2 is below 10 μM, which means that this compound has strong HeLa cell growth inhibiting effect. As a comparison, the IC₅₀ for Compound 1 is about 10 times higher.

TABLE 4
IC₅₀ Values of Four New Compounds at 72 h on HeLa cells

| Compound | IC₅₀ (μM) |
|---|---|
| 1 gpl-15 | 95.6 ± 5.5 |
| 2 gpl-26 | 9.5 ± 0.2 |

While there have been described and pointed out fundamental novel features of the invention as applied to a preferred embodiment thereof, it will be understood that various omissions and substitutions and changes, in the form and details of the embodiments illustrated, may be made by those skilled in the art without departing from the spirit of the invention. The invention is not limited by the embodiments described above which are presented as examples only but can be modified in various ways within the scope of protection defined by the appended patent claims.

What is claimed is:

1. A compound, having a structural as follows:

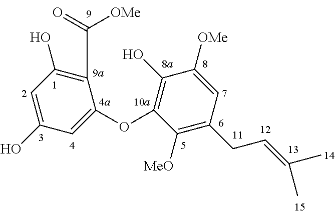

2. A method of inducing apoptosis in tumor cells, comprising a step of administering to a mammalian subject a therapeutically effective amount of the compound of claim 1.

3. The method according to claim 2, wherein said mammalian subject is a human patient suffering from cancer.

4. A pharmacological composition, comprising a compound of claim 1 and a pharmaceutically acceptable carrier.

5. The pharmacological composition according to claim 4, wherein the pharmacological composition is formulated in a dosage form selected from a group consisting of tablet, capsule, and injection.
